(19) United States
(12) Patent Application Publication
(10) Pub. No.: US 2017/0226541 A1
(43) Pub. Date: Aug. 10, 2017

(54) METHOD OF IMPROVING SOLVENT TOLERANCE IN MICROBES

(71) Applicants: Nanyang Technological University, Singapore (SG); National University of Singapore, Singapore (SG)

(72) Inventors: Jamie Hinks, Singapore (SG); Stefan Wuertz, Singapore (SG)

(21) Appl. No.: 15/502,747

(22) PCT Filed: Aug. 21, 2015
(86) PCT No.: PCT/SG2015/050272

§ 371 Date: Feb. 8, 2017

Related U.S. Application Data
(60) Provisional application No. 62/040,852, filed on Aug. 22, 2014.

(51) Int. Cl. C12P 7/16; C12N 1/04; C12N 15/01

(52) U.S. Cl. CPC C12P 7/16; C12N 15/01; C12N 1/04; C12R 1/19

(57) ABSTRACT

The invention relates generally to a method of improving solvent tolerance in microbes, and in particular, to a method of improving bio-butanol titre in a fermentation process due to improved solvent tolerance in microbes. The method of improving butanol titre in a fermentation process involves the addition of a membrane insertion molecule comprising a conjugated oligoelectrolyte, particularly an oligo-polyphenylene vinylene conjugated oligoelectrolyte.

### CROSS-REFERENCE TO RELATED APPLICATION

This application claims the benefit of priority of U.S. Provisional Patent Application No. 62/040,852, filed Aug. 22, 2014, the contents of which being hereby incorporated by reference in its entirety for all purposes.

### TECHNICAL FIELD

The invention relates generally to a method of improving solvent tolerance in microbes, and in particular, to a method of improving bio-butanol titre in a fermentation process due to improved solvent tolerance in microbes.

### BACKGROUND

Biofuels are becoming increasingly important to meet today's society's growing energy demand and simultaneous but conflicting need to reduce carbon emissions. Being a drop-n fuel, butanol is attractive because it can be readily mixed with gasoline without modification to current engines and can be transported in current oil and gas pipelines/infrastructure. Compared to ethanol, butanol has a higher energy density, is less hygroscopic, less volatile and has a lower flashpoint. Isomers of butanol include butan-1-ol, butan-2-ol, 2-methylpropan-1-ol, and 2-methylpropan-2-ol.

Bio-butanol is produced anaerobically by a number of native butanol-producing organisms in what is known as the acetate butanol ethanol (ABE) fermentation process. Clostridium acetobutylicum and C. beijerinckii are archetypical representative of ABE fermentative organisms. One problem with bio-butanol production is that the titre is low, the cause of which is predominantly due to the toxicity of butanol and the other solvents to cells. The nature of butanol and solvent toxicity arises from its hydrophobic properties and its ability to disrupt microbial membranes. Bio-butanol titres of 1.3 g L⁻¹ are recognised as an upper limit tolerated by ABE organisms.

Various strategies for increasing bio-butanol production have been explored, including process optimizations with natural butanol-producing strains, genetic engineering natural producers to, for example, promote greater product specificity, and genetic engineering the butanol synthetic pathway into non-producing organisms, typically Escherichia coli. However, butanol is inhibitory to bacterial growth and growth rates of C. acetobutylicum are reduced by 50% at butanol concentrations of just 1.0% v/v. Regardless of the success of these strategies to improve bio-butanol production, unless the tolerance of cells to butanol is increased, bio-butanol titres can never realize their full potential. Large savings in production costs can be realized from only very small increases in bio-butanol titre, however, and there is considerable commercial value in increasing microbes tolerance to solvents.

Therefore, there remains a need to provide for a method that improves solvent tolerance in microbes which ultimately leads to improved bio-butanol titres during fermenation.

### SUMMARY

Present inventors have surprisingly found that a conjugated oligo-electrolyte which spontaneously inserts into microbial membranes can enhance butanol tolerance in non-butanol-producing microorganisms such as E. coli. An enhanced growth of microorganisms with presently disclosed method of treatment at concentrations ranging from 0.85 to 3.5% v/v butanol has been achieved. The effect of present treatment method is most pronounced at 3.5% v/v butanol where the growth rate of the treated cells is 3 times that of untreated cells (0.095 vs 0.032 h⁻¹), representing a growth rate of about 13 and 4%, respectively, relative to a control (see FIG. 1). For comparison, a growth rate of 0.18 h⁻¹ was recently reported for an engineered butanol tolerant strain but this was not able to grow above 1.8% v/v, and a number of strains of E. coli were reported as being unable to grow at butanol concentrations above 2% v/v.

According to a first aspect of the disclosure, there is provided a method of improving solvent tolerance in microbes. The method includes modifying the cellular membrane of the microbes. The modifying step includes contacting the microbes with a membrane insertion molecule, wherein the membrane insertion molecule comprises a conjugated oligo-electrolyte.

According to a second aspect of the disclosure, there is provided a method of improving bio-butanol titre in a fermentation process. The method includes modifying a cellular membrane of the microbes used in the fermentation process. The modifying step includes contacting the microbes with a membrane insertion molecule, wherein the membrane insertion molecule comprises a conjugated oligo-electrolyte. The method further includes allowing the microbes to grow in an environment comprising butanol.

In various embodiments of the first and second aspects, the membrane insertion molecule comprising the conjugated oligo-electrolyte comprises an oligo-polyphenylene vinylene conjugated oligo-electrolyte.

### BRIEF DESCRIPTION OF THE DRAWINGS

In the drawings, like reference characters generally refer to the same parts throughout the different views. The drawings are not necessarily drawn to scale, emphasis instead generally being placed upon illustrating the principles of various embodiments. In the following description, various embodiments of the invention are described with reference to the following drawings.

FIG. 1 shows a comparison between treated and untreated E. coli cells, showing the difference in butanol tolerance; the protective effect of presently disclosed compound (OPV COE) at a concentration of 3.5% v/v butanol is particularly evident with the specific growth rate three times that of the untreated control.

FIG. 2 shows diffusion coefficients in supported membranes treated with 10 μM COE1-5C and subjected to butanol challenges of 0.85, 1.75, and 3.50% v/v butanol.

FIG. 3 shows representative time-evolution snapshots of the perturbation of an untreated lipid bilayer system by (A) 5% butanol (mol/mol), and (B) 1.5% butanol (mol/mol), as predicted by molecular dynamics simulation; at 0, 50, 100 and 200 ns. Butanol molecules are illustrated in red, water in gray and POPE/POPG in green.

FIG. 4 shows mean square displacement over time of fluorescently-labeled phospholipid biomarker DHPE-Atto647N within a glass supported lipid bilayer of E. coli total lipid extract with four OPV COE inclusions; 4F-DSBN⁺, DSSN⁺, DSBN⁺ and COE1-5C.

FIG. 5 shows (A) spatially resolved map of average POPE/POPG bilayer thickness with and without MIM (COE1-5C and DSSN⁺) following 200 ns simulation time; The total area shown in each box is 6 nm×6 nm; (B) Lipid bilayer thickness for the control system and with COE1-5C and DSSN⁺, with averaged across the entire 200 ns simulation period.

FIG. 6 shows representative time-evolution snapshots of the perturbation of a lipid bilayer system in 5% butanol (mol/mol) treated with (A) COE1-5C and (B) DSSN+, as predicted by molecular dynamics simulation; at 0, 50, 100 and 200 ns. Butanol molecules are illustrated in red, MIMs in blue, water in gray and POPE/POPG in green.

FIG. 7 shows the extent of membrane depolarization of E. coli K12 treated with 10 μM COE1-5C in response to increasing butanol challenge against a control.

FIG. 8 shows the respective chemical structure of 4F-DSBN+, DSSN+, DSBN+, and COE1-5C.

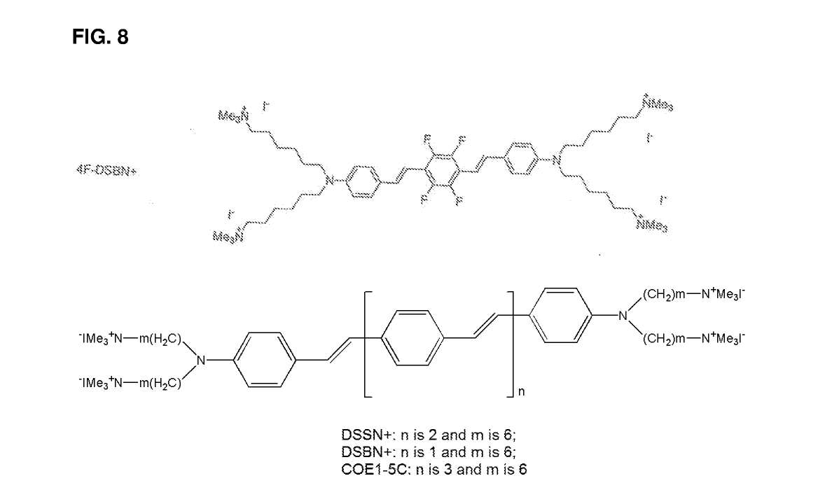

### DESCRIPTION

The following detailed description refers to the accompanying drawings that show, by way of illustration, specific details and embodiments in which the invention may be practised. These embodiments are described in sufficient detail to enable those skilled in the art to practise the invention. Other embodiments may be utilized and structural and chemical changes may be made without departing from the scope of the invention. The various embodiments are not necessarily mutually exclusive, as some embodiments can be combined with one or more other embodiments to form new embodiments.

Low butanol tolerance stems from the membrane disrupting effects of the acetone, butanol and ethanol mixture typically produced in ABE process (which is named after the initials of the solvents produced). Long chain alcohols (>C3) disrupt hydrogen bonding between the acyl chains of the phospholipid bilayer. The increased membrane disorder fluidizes the membrane causing it to deviate from the homeoviscous state required for normal membrane function.

Accordingly, to improve butanol tolerance in microbes, cellular membranes need to be stabilized.

Thus, according to a first aspect of the present disclosure, there is disclosed a method of improving solvent tolerance in microbes. The method includes modifying a cellular membrane of the microbes. The modifying step includes contacting the microbes with a membrane insertion molecule, wherein the membrane insertion molecule comprises a conjugated oligo-electrolyte

Membrane insertion molecules (MIMs) are molecules that modify cell function or stability by intercalating into the phospholipid bilayer of microbial membranes. Traditionally, they have found use as antimicrobials but it has been discovered that there is huge potential to apply MIMs to enhance a range of biotechnologies, such as in present case, to improve solvent tolerance in microbes.

On account of the localized changes in membrane order observed previously, it is herein hypothesised that it is possible to increase membrane stability under conditions of high solvent concentration by use of oligo-polyphenylene vinylene (OPV) conjugated oligo-electrolytes (COEs). Molecular length and charge distribution (particularly how charge affects their range of motion in the bilayer) are the two main structural determinants of toxicity for the OPV COEs investigated. It should therefore be possible to not only mitigate membrane perturbation but also enhance membrane stability under extreme environmental conditions through intelligent molecular design, which accounts for particular features of the target membrane such as phospholipid profile and acyl chain configuration.

Exogenously conferring solvent tolerance in microbes addresses the biotechnological puzzle of how to match microbial physiology with favorable bioprocess conditions. Additionally, supplementing a system with MIMs to confer solvent tolerance is compatible with using complex, undefined microbial consortia for bio-butanol production, such as in conversions using complex organic feeds. In this disclosure, MIMs would confer solvent tolerance to the whole community including organisms that do not directly produce butanol but, nonetheless, carry out reactions critical to bioprocess stability.

In this disclosure, it is demonstrated that increased solvent tolerance can be achieved by supplementing a monoculture of E. coli with MIMs. Such a practice would therefore be directly compatible with E. coli strains genetically engineered for increased bio-butanol production, although the concept will find utility in both mono- and mixed-cultures of other butanol-producing communities. It is herein established that butanol tolerance stems from the ability of MIMs to stabilize the phospholipid bilayer and herein proposed a model to account for this observation.

Present approach relies on a chemical agent (i.e. the OPV COE) to induce solvent tolerance in microorganisms instead of traditional culture-based or genetic and metabolic manipulation. The main advantage is that this is a rapid, non-selective intervention to induce immediate solvent tolerance in a particular community. It can be used in response to changing conditions or to induce solvent tolerance at a particular point in the process of interest. In this way, its action is analogous to that of a therapeutic agent, but one designed specifically for microbes.

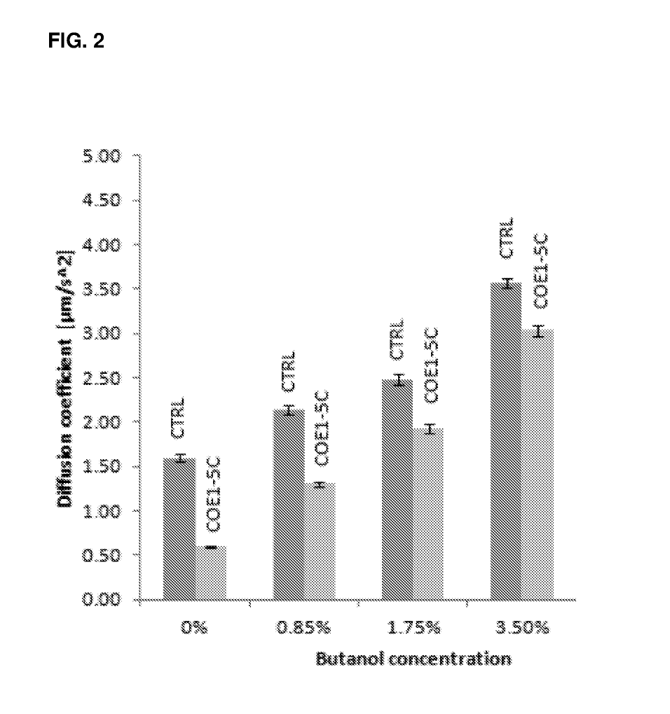

The mechanism of action that imparts solvent tolerance to the cells may be explained in terms of the effect of COE insertion into the cellular membrane and their subsequent effect on the diffusion parameters of phospholips. FIG. 2 shows that the diffusion coefficients in model membranes treated with COE1-5C are much lower than in control systems, a trend that is maintained as butanol concentration is increased. The ability of COEs to counter the fluidizing effects of butanol is likely to underlie COEs protective effect on biological membranes.

Additionally, cells treated with COE1-5C partition into butanol less readily than untreated cells. Table 1 shows the MATH value determined from a normalised cell butanol partitioning assay and quantitatively shows the tendency for COE1-5C treated cells to partition into the butanol phase is less than half that of untreated cells. COE-15C treatment of bacterial cells therefore reduces hydrophobic interactions between the cell envelope and butanol, thus effectively reducing cell exposure to butanol.

TABLE 1
MATH value of untreated E. coli K12
and those treated with 10 μM COE1-5C.

| | MATH Value |
|---|---|
| Untreated | 0.35 (±0.073) |
| Treated | 0.17 (±0.027) |

In various embodiments, the membrane insertion molecule comprising the conjugated oligo-electrolyte comprises an oligo-polyphenylene vinylene conjugated oligo-electrolyte. The oligo-polyphenylene vinylene oligo-electrolyte comprises a central backbone of 1, 2, 3, 4, or 5 benzene rings. Preferably, the oligo-polyphenylene vinylene oligo-electrolyte comprises a central backbone of 5 benzene rings. The conjugated aromatic backbone is terminated at each end by two pendant alkyl chains. The alkyl chains may consist of 1, 2, 3, 4, 5, 6, 7, 8, 9, or 10 carbon atoms in length and are functionalized with tertiary ammonium group whose charge is balanced by iodine counter ions, for example. This functionality renders the molecule amphiphilic and imparts water solubility to the molecule. Upon mixing with microbial cells, the molecule associates preferentially with the microbial membranes rather than in the aqueous phase. This intimate association of the molecule with the microbial envelope affects the chemistry of microbial membranes and promotes ordering of the acyl chains in the lipid bilayer, and it is this property that counters the membrane disordering effects of solvents.

In various embodiments, the oligo-polyphenylene vinylene conjugated oligo-electrolyte comprises formula (I):

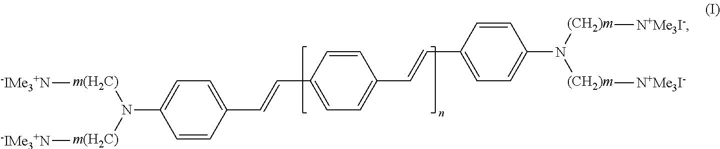

wherein m is an integer from 1 to 10 and n is an integer from 1 to 5, where Me represents a methyl group.

For example, in formula (I) m may be an integer from 1 to 6 and n may be 1, 2, or 3.

In one embodiment, the oligo-polyphenylene vinylene conjugated oligo-electrolyte comprises the following structure (COE1-5C):

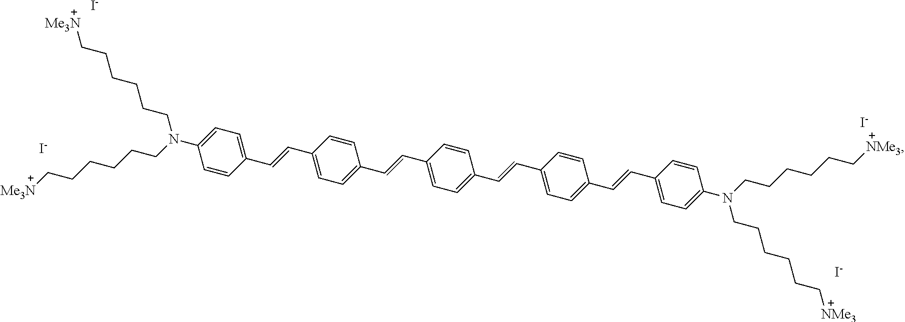

wherein in formula (I) m is 6 and n is 3.

A second aspect of the present disclosure is an extension of the first aspect, whereby a method of improving bio-butanol titre in a fermentation process is disclosed. The method includes modifying a cellular membrane of the microbes used in the fermentation process. The modifying step includes contacting the microbes with a membrane insertion molecule, wherein the membrane insertion molecule comprises a conjugated oligo-electrolyte. The method further includes allowing the microbes to grow in an environment comprising butanol.

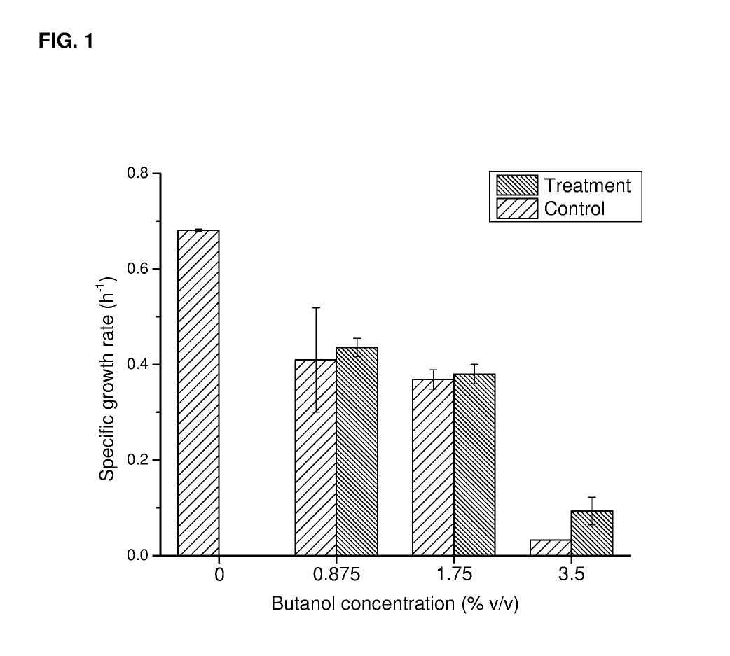

As mentioned in earlier paragraphs as well as described in the examples section below, an enhanced growth of microorganisms with presently disclosed method of treatment at concentrations ranging from 0.85 to 3.5% v/v butanol has been achieved, although higher concentrations of butanol may also be used, such as 0.85 to 8% v/v butanol. The effect of present treatment method is most pronounced at 3.5% v/v butanol where the growth rate of the treated cells is 3 times that of untreated cells (0.095 vs 0.032 h⁻¹), representing a growth rate of about 13 and 4%, respectively, relative to a control (see FIG. 1). For comparison, a growth rate of 0.18 h⁻¹ was recently reported for an engineered butanol tolerant strain but this improvement was not demonstrated above 1.2% v/v butanol, and a number of strains of E. coli were reported as being unable to grow at butanol concentrations above 2% v/v butanol. Growth at concentrations greater than 3.5% v/v butanol is rare in unmodified organisms.

Thus, in various embodiments, the method of the second aspect includes allowing the microbes to grow in an environment comprising 0.85 to 8% v/v butanol, preferably 2 to 3.5% v/v butanol, more preferably 3.5% v/v butanol.

In order that the invention may be readily understood and put into practical effect, particular embodiments will now be described by way of the following non-limiting examples. In this context, unless otherwise indicated, concentrations of butanol described in the experiments are in % v/v while concentrations of butanol described in modelling studies are in % mol/mol.

### Examples

In the examples described herein, fluidization of microbial membranes by butanol limits biobutanol production titres. In vive and in vitro techniques within in silico framework have been applied to achieve bacterial solvent tolerance. Single molecule tracking of a model bilayer showed that a 5-ringed oligo-polyphenylene vinylene (OPV) conjugated oligo-electrolyte (COE) reduced phospholipid diffusion more than 3- and 4-ringed COEs and increased the growth rate of E. coli K12 at inhibitory butanol concentrations. Conferring butanol tolerance exogenously complements genetic modifications and is additionally compatible with complex and undefined microbial consortia for biobutanol production. Molecular dynamic simulations indicated that the COE aromatic backbone acts as a hydrophobic tether for glycerophospholipid acyl chains enhancing bilayer integrity at high butanol concentrations. The 5-ringed COE mitigated E. coli K12 membrane depolarization, suggesting that improved growth in the presence of butanol results from enhanced bilayer stability.

### EXPERIMENTAL SECTION

Materials

(DSSN+) was synthesized and characterized according to Garner et al (L. E. Garner, J. Park, S. M. Dyar, A. Chworos, J. J. Sumner, G. C. Bazan, Journal of the American Chemical Society 2010, 132, 10042-10052). The method for synthesizing (DSBN+) and (4F-DSBN+) is described in Woo et al (H. Y. Woo, B. Liu, B. Kohler, D. Korystov, A. Mikhailovsky, G. C. Bazan, Journal of the American Chemical Society 2005, 127, 14721-14729) while COE1-5C synthesis is described in Hou et al (H. Hou, X. Chen, A. W. Thomas, C. Catania, N. D. Kirchhofer, L. E. Garner, A. Han, G. C. Bazan, Advanced Materials 2013, 25, 1593-1597). The uptake of these molecules into biological membranes is well documented (J. Hinks, Y. Wang, W. H. Poh, B. C. Donose, A. W. Thomas, S. Wuertz, S. C. Loo, G. C. Bazan, S. Kjelleberg, Y. Mu, T. Seviour, Langmuir 2014, 30, 2429-2440).

Organism Selection

Escherichia coli K12 (ATTC 10798) was used in all studies presented herein and was selected as a representative industrially convenient organism and commonly modified strain for enhanced butanol production and synthesis. An additional experimental convenience of choosing this organism is that lipid extracts exist as do paramaterised molecular dynamic frameworks allowing for a range of complementary investigations.

Single Particle Tracking Analysis of Lipid Bilayer Extracts from E. coli

SLB formation, MIM treatment: A 3 mg/ml chloroform solution of total E. coli lipid extract (Avanti Polar Lipids, Alabaster, Ala.) was mixed with DHPE-Atto647N (Atto-Tec, Slegen, Germany) to 0.001% mol in a round bottom flask produce large unilamellar vesicles (LUVs). Chloroform was evaporated using a rotary evaporator for 2 hours at 65° C. The lipid film was resuspended in 10 mM HEPES and 150 mM NaCl, pH 7.4, vortexed for 10 min and sonicated in a bath sonicator until a clear solution was obtained. One hundred μl of such a prepared LUVs solution were deposited in a custom made plastic holder, glued to a coverslip, and incubated for 60 min at 65° C. Prior to use, coverslips were cleaned by 30 min sonication in 2% Hellmanex (Hellma Analytics, Millheim, Germany), followed by thorough rinsing with deionized (DI) water. Un-fused vesicles were removed by 10× washing with 400 μl of 10 mM Hepes, 150 mM NaCl, 3 mM EDTA, pH 7.4 buffer. Calculated volumes of COE1-5C, DSSN+, DSBN+ and 4F-DSBN+ (0.1 mM) were added directly to the chamber to final concentrations of 1 μM, mixed and allowed to equilibrate for 5 min prior to measurement.

Data acquisition: For total internal reflection microscopy (TIRF) imaging, a modified Nikon Eclipse TE2000U TIRF setup was used, equipped with a 100× 1.49NA objective (Nikon, Japan). Laser light from a 638 nm LED laser was passed through a 660LP dichromic mirror (Chroma Technology Corp, Bellows Falls, Vt.) and used for sample illumination under the TIR angle. Emitted light was filtered using a 738/75 excitation filter and collected on a back-illuminated EMCCD camera chip (Andor iXON897, 512x512px, Andor Technology, USA). For each condition, 4 to 6 datasets of 4000 frames at 97 fps were collected from different regions of the membrane. Immobile particles and contaminants were bleached prior to measurement.

Data analysis: A Mosaic Particle Tracker ImageJ plugin was used to localize the centroid of single molecule signals and link them to trajectories. TrackArt software was used to filter spurious trajectories and extract mean square displacements and diffusion coefficients. Analysis showed two distinct diffusing populations (fast- and slowly-diffusing), hence two-population models were used to extract diffusion coefficients. The slowly-diffusing population occurs most likely due to marker interaction with the surface. Since the diffusion coefficient of slowly-diffusing population does not change significantly during conducted experiments, and is over an order of magnitude lower than for fast population, only the fast diffusion coefficient component is presented in this example.

Molecular Dynamic Simulations of OPV COE Molecules and POPE/POPG

The palmitoyloleoylphosphatidylethanolamine (POPE) and palmitoyloleoylphosphatidylglycerol (POPG) lipid bilayer was applied in this example to mimic a negatively charged, generic, Gram-negative bacterial cell membrane. The bilayer consisted of 96 POPE (75 mol %), 32 POPG (25 mol %), and ˜4000 water molecules. The system was ionized with 190 mM NaCl.

Two types of OPV COE molecules, DSSN+ and COE1-5C, were simulated in the model membrane. In each system, nine OPV COE molecules were added into the lipid bilayer, representing lipid:OPV COE ratios of 128:9. OPV COE molecules were fully inserted into the POPE/POPG bilayer as initial structures, and overlapping lipids removed. MD simulations were applied over 200 ns to obtain the equilibrium state of OPV COE molecules inside membrane. Counter ions of Cl⁻ or Na⁺ were added to neutralize system charge. Furthermore, four concentrations (0%, 0.75%, 1.5%, 5% mol/mol) of butanol were applied outside the membrane. The same series of butanol systems were used outside the bilayer without OPV COE molecules as control. Three trajectories of each simulation system were monitored for 200 ns with different initial orientations of butanol molecules. Topologies of POPE/POPG lipids and DSSN+ were chosen from a previous study. Butanol and COE1-5C molecules were built from Automated Topology Builder (ATB).

All simulations were run using the Gromacs 4.5 package. Gromos53a6 force-field parameters were applied. The SPC (simple point charge) model was used for water molecules. A periodic boundary condition (PBC) was applied to the system. The V-rescale coupling was used to maintain the temperature at 300 K. Semi-isotropic Parrinello Rahman pressure coupling with a pressure of 1.0 atm was used in this example. The pair-list of non-bonded interactions was recalculated every 10 time-steps with a pair-list cutoff distance of 12 Å used. The Particle Mesh Ewald method was used for full evaluation of long-range electrostatic interactions. The LINCS routine with a tolerance of 104 was used to constrain all bond lengths with hydrogen atoms in all simulations. Atomic coordinates were saved every 1 ps for subsequent data analysis.

Simulation Analysis

Bilayer thickness and deuterium order parameter (Scd) of the carbon tail were applied to asses POPE/POPG lipid bilayer state with or without OPV COE insertion. The GridMAT-MD package was used to analyze the bilayer thickness by measuring the distance of the phosphate groups in the upper and lower leaflets. Deuterium order parameter was calculated by the GROMACS software package.

Butanol Challenge

Butanol challenge was assessed by using a microdilution format modified standard toxicity test based on the method described by Wiegand et al (I. Wiegand, K. Hilpert, R. E. W. Hancock, Nat. Protocols 2008, 3, 163-175). Cultures of E. coli K12 (ATTC 10798) grown in LB broth were harvested at late log phase and diluted to 1×10⁶ cfu ml. One hundred μl of the suspension were inoculated into 96 well plates with each well containing an equal volume of LB broth and spiked with the following concentrations of 1-butanol: 0, 1.75, and 3.5 v/v butanol in a 96 well plate achieving a final inoculum density of 5×10⁵ and a final butanol concentration of 0, 0.85, 1.75, and 3.5% (v/v). Tests were run in triplicate with and without COE1-5C (10 μM) and DSSN+ (5 μM) using the appropriate sterility and growth controls. Specific growth rates were expressed as an average of all replicates.

Membrane Polarisation

Cultures of E. coli K12 grown in LB broth were harvested at late log phase and diluted to 5×10⁵ cfu ml. Cells were centrifuged at 5000×g for 5 min and washed in phosphate buffered saline (PBS). The process was repeated three times and the pellet finally resuspended in PBS. Two-hundred μl of the cell suspension were inoculated into 96 well plates, 2 μL of the depolarization detecting probe 3,3-diethylthiodicarbocyanine (DiSC3) iodide was added to the treatment wells and to half of these, 10 μM COE1-5C. The cells were allowed to incubate for around 30 min and the fluorescence intensity at 670 nm was measured after excitation at 620 nm until a stable base line was reached. Zero, 1.75, or 3.5% (v/v) 1-butanol was added to the test well and the increase in fluorescence intensity was taken at 1 min intervals (Ex=λ620, Em=Δ670) as a bottom reading with optimal gain to determine the degree of membrane polarization. Buffer, dye, and COE1-5C blanks were monitored and the data corrected accordingly.

Modified Microbial Adhesion to Hydrocarbon (MATH) Test

To test for cell surface modifications brought about by COE1-5C that might affect hydrophobicity, E. coli K12 cells treated with COE1-5C (10 μM) were tested for their affinity for either the butanol or aqueous phase against an untreated control. The tendency of E. coli to partition into the butanol phase or remain in the aqueous phase was assessed optically by measuring the reduction in turbidity (OD600) of tubes containing a saturated mixture of butanol (1.5 ml) and buffer (18.5 ml). The tests were carried out with appropriate blanks and both positive and negative controls. MATH tests were carried out in triplicate and expressed as the quotient of the difference between the OD600 at the start and end of the experiment and the OD600 at the end of the experiment.

Results and Discussion

Cell Membrane Disruption by Butanol

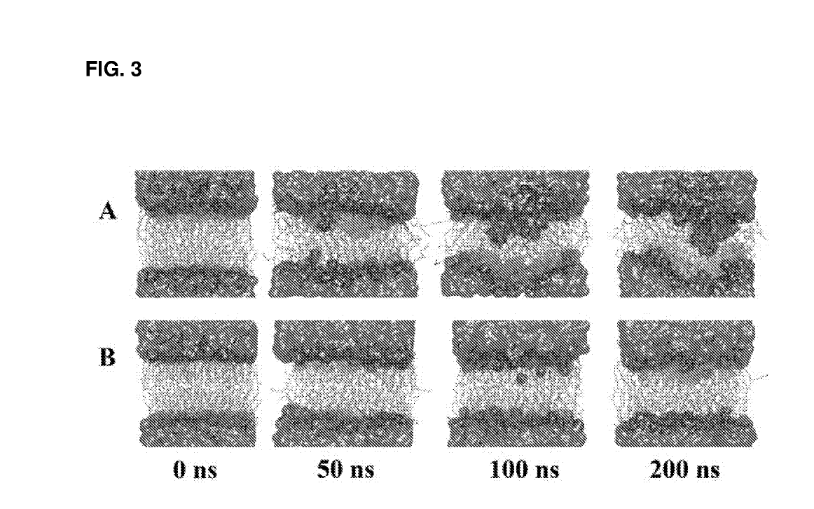

The fluidization of a generic, Gram-negative model membrane bilayer composed of (3:1) palmitoyloleoylphosphatidylethanolamine (POPE)/palmitoyloleoylphosphatidylglycerol (POPG) by butanol was simulated (FIG. 3). In the presence of 5% butanol (mol/mol), the bilayer is disrupted due to hydrophobic interactions between the acyl chains of the phospholipids and butanol (FIG. 3(A)). Partial solublisation of the phospholipid bilayer effectively ensues, with the formation of water channels through the bilayer as water molecules supplant displaced phospholipids. In 1% butanol (mol/mol) a similar response was observed, but with a smaller proportion of acyl chains dissolving into the butanol phase (FIG. 3(B)), indicating that lipid bilayer disruption is proportional to the concentration of solvent.

Potential for Membrane Insertion Molecules to Mitigate Bilayer Disruption by Butanol

Given that solvents extract phospholipids from the bilayer (FIG. 3), this effect could be mitigated by membrane insertion molecules (MIMs), specifically oligo-polyphenylene vinylene COEs, which can affect membrane order after they spontaneously intercalate the lipid bilayer. After insertion of OPV COEs into the membrane, the n-conjugated aromatic backbone is thought to align with the fatty-acid acyl chains of phospholipids with the cationic pendant moiety oriented toward the phosphate head groups. Thus, it is herein postulated that the n-conjugated aromatic backbone of OPV COEs would promote hydrogen bonding between the fatty acid acyl chains within the bilayer, effectively acting as a tether and permitting lipids to remain in an ordered lamellar phase upon exposure to butanol.

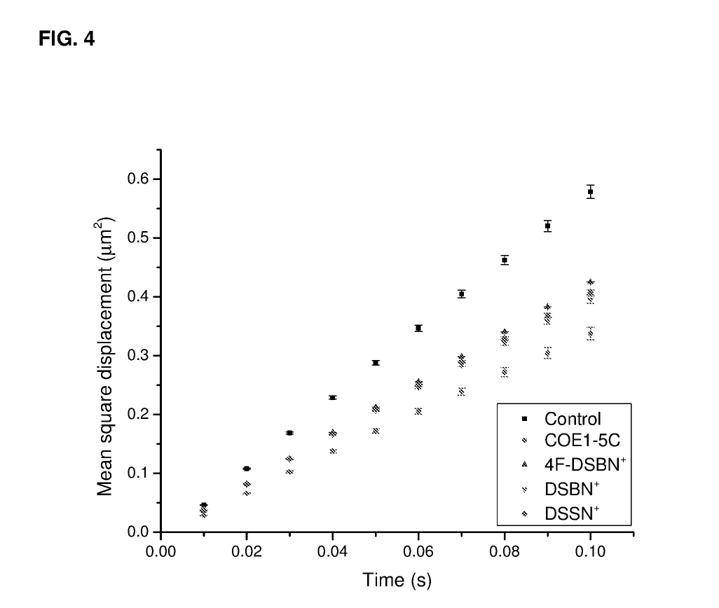

To support the hypothesis that OPV COEs can help maintain membrane integrity by increasing hydrophobic interactions within lipid bilayers, single molecule tracking (SMT) was undertaken on a fluorescently-labeled phospholipid membrane marker (DHPE-Atto647N) within a model supported-bilayer system consisting of E. coli total lipid extract, in the presence and absence of four OPV COEs; 4F-DSBN+, DSSN+, DSBN+ and COE1-5C. The mean square displacement (MSD) and diffusion coefficient of the fluorescently labeled lipid bilayer marker within the model bilayer system was determined (FIG. 4). All OPV COEs tested reduced the MSD, and hence diffusion coefficient (FIG. 2), of the marker phospholipid through the E. coli lipid-extract bilayer, indicating reduced molecular mobility and increased bilayer rigidity. These results indicate that the five-ringed molecule, COE1-5C, increased bilayer stiffness to the greatest degree. DSBN+ and DSSN+ reduced phospholipid MSD by similar amounts to one another but to a lesser degree than the five-ringed structure. The lipid bilayer was stiffened the least amount by 4F-DSBN+, consistent with a previous observation that fluorination of the central aromatic ring allows a greater range of movement between cationic pendant chains and aromatic backbone, thus preserving lipid bilayer fluidity.

Relative toxicities of DSSN+ and DSBN+ are positively correlated with the extent of mismatch between the length of the hydrophobic molecular backbone and bilayer thickness. Membrane insertion of the shorter, 3-ringed DSBN+ thinned the membrane more than the 4-ringed DSSN+, perturbing it to a greater extent, and was correspondingly more toxic. In this example, the objective is to mitigate the toxic effect of organic solvents on biological membranes. To avoid synergistic membrane disruption in systems containing both MIMs and solvents, MIMs that were the least likely to perturb membranes, that is, DSSN+×(which was previously shown to maintain the natural thickness of membranes) and COE1-5C, were selected. The insertion of COE1-5C, whose molecular length (≈5 nm) is greater than the thickness of the bilayer (≈4 nm), would be expected to initiate membrane perturbation because of hydrophobic mismatch. However, positive hydrophobic mismatch perturbs lipid bilayers less than instances of negative mismatch—where the length of the insertion molecules is shorter than the thickness of the bilayer. The experimental concentration of COE1-5C used in this study (10 μM) is much lower than its minimum inhibitory concentration (MIC). Toxicity tests conducted on E. coli K12 revealed the MIC for COE1-5C to be 128 μmol L-1. This is considerably greater than for DSSN+, DSBN+ and 4F-DSBN+, further supporting the hypothesis that membrane thinning is a potent toxicity determinant. COE1-5C is encouraging as a candidate to mitigate membrane destabilization by organic solvents due to its low intrinsic toxicity and greater ability to impart rigidity to the lipid bilayer than other OPV COEs (FIG. 4).

Consistent with the molecular dynamic predictions (FIG. 3(B)), the growth of E. coli was inhibited at concentrations as low as 0.85% v/v butanol, and growth was severely inhibited at 3.5% v/v butanol and cell growth at concentrations above this is rare in unmodified organisms (FIG. 1). E. coli K12 was subjected to butanol challenge in the presence of DSSN+ and COE1-5C, with butanol concentrations ranging between 0 to 3.5% (v/v). Whilst no protective effect was shown for DSSN+ (data not shown), COE1-5C increased the growth rate of E. coli K12 in all instances of butanol challenge. The greatest protective effect was observed at 3.5% (v/v) butanol concentration as evident from a threefold increase in the specific growth rate from 0.032 (±0.0003) to 0.094 (±0.029) h⁻¹ (FIG. 1). A reduction in growth rate is in keeping with previous studies and the growth rate achieved here under 3.5% v/v butanol challenge of around 0.09 h⁻¹ is comparable with growth rates reported elsewhere of 0.18 h⁻¹, for an E. coli strain improved for butanol tolerance using random mutagenesis, but under a more moderate butanol challenge of 1.2% v/v butanol (H. Zhang, H. Chong, C. Ching, H. Song, R. Jiang, Applied Microbiology and Biotechnology 2012, 94, 1107-1117).

Treatment by COE1-5C Preserves Homeoviscosity of Lipid Bilayer Under Butanol Challenge

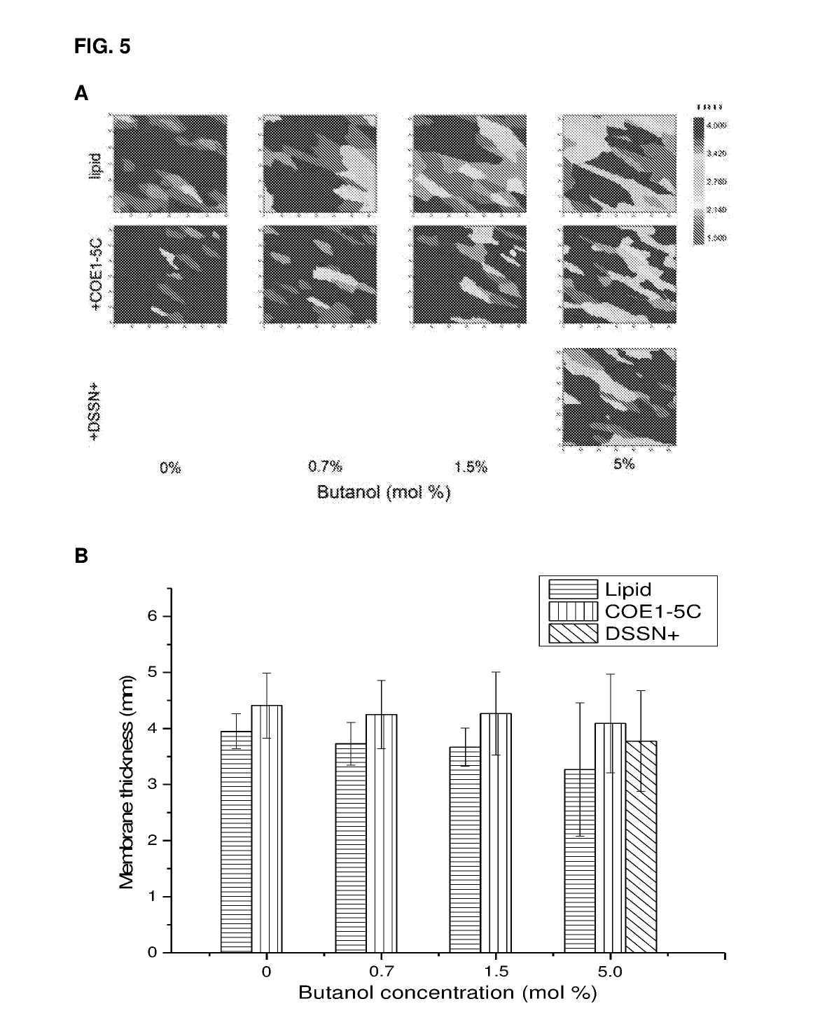

Single molecule tracking was undertaken on a supported bilayer of E. coli lipid extract with and without COE1-5C in media with four different butanol concentrations: 0, 0.85, 1.75, and 3.5% (v/v) (FIG. 1). The diffusion coefficient of the membrane marker increased with butanol concentration both with and without COE1-5C, indicative of membrane disorder. Treatment with COE1-5C, however, mitigated the bilayer disordering tendency of butanol, with 62, 38, 22 and 15% reductions in MSD for 0, 0.85, 1.75, and 3.5% (v/v) butanol, respectively (FIG. 5(A)).

MD Simulations Indicate Reduced Dissolution of Lipids into the Butanol Phase when Treated with COE1-5C

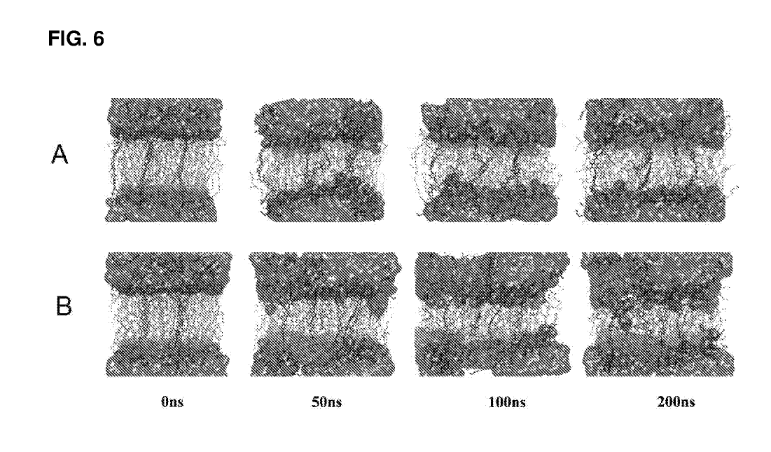

The effect of COE1-5C on the model POPE/POPG lipid bilayers was simulated in the presence of butanol to investigate whether the enhanced growth rates observed at high butanol concentrations (i.e., 5% mol/mol) can be explained by reduced membrane perturbation. The extent of acyl chain extraction from the lipid bilayer was lower when COE1-5C was present (FIG. 6) than not (FIG. 3(A)). Formation of water channels was not observed in any of the simulations when COE1-5C was present. A spatially resolved map of bilayer thickness (FIG. 5) shows that in the absence of COE1-5C, phospholipid extraction by butanol led to a reduction in membrane thickness that was proportional to butanol concentration with localized depletion in the thickness over 60% recorded at various positions after exposure to 5% butanol (mol/mol) (FIG. 5(B)). Furthermore, the extremely large variance associated with the observations of bilayer thickness for the untreated membrane in the presence of 5% butanol (mol/mol) solution indicates severe membrane disruption under these conditions.

Bilayer disruption by butanol, however, was mitigated by COE1-5C. A more consistent average bilayer thickness was maintained throughout the simulation in the presence of COE1-5C (FIG. 5(A)) and the associated variance much smaller than those in the absence of COE1-5C indicating a more intact membrane.

In a previous study, it is shown that the insertion of several OPV COEs of different length into the lipid bilayer distorted the membrane according to the degree of hydrophobic mismatch—the difference between molecular length and bilayer thickness. In the current example, the effect on the lipid bilayer of COE1-6C insertion, a molecule with a 5-ringed aromatic backbone and a total length (≈5 nm) greater than the bilayer thickness (≈4 nm) was investigated. The simulations indicated that in the absence of butanol, COE1-5C, as expected, increased the average membrane thickness by approximately 15% following a 200 ns simulation period. Additionally, the model indicates that the average extent of bilayer disruption upon exposure to butanol is dramatically reduced when COE1-5C is present. Average membrane thickness was only reduced by 0.2 nm or 5% in the presence of COE1-5C while in the absence of the membrane insertion molecule the membrane thickness decreased by approximately 18%.

To determine whether the ability to mitigate membrane fluidization by butanol correlates with the molecular length, the simulation in 5% butanol (mol/mol) was repeated for DSSN+, the shorter (≈4 nm) MIM shown previously to be the least disruptive to membranes. Water pores within the membrane were induced by 5% butanol (mol/mol) by the end of the 200 ns simulation even with DSSN+ incorporated in the lipid bilayer (FIG. 5(A)). Mitigation of butanol toxicity can therefore be described for COE1-5C but not DSSN+. The fact that no growth of E. coli K12 was observed in incubations when DSSN+ and butanol were present supports this observation (data not shown).

In Vivo Data Supports Observations of MD Simulations and SMT

Living cells maintain an electrical potential across their membranes. Disruption of this potential by, for example, either MIMs or solvents, would result in cell death by a mechanism independent of, or preceding, water pore formation.

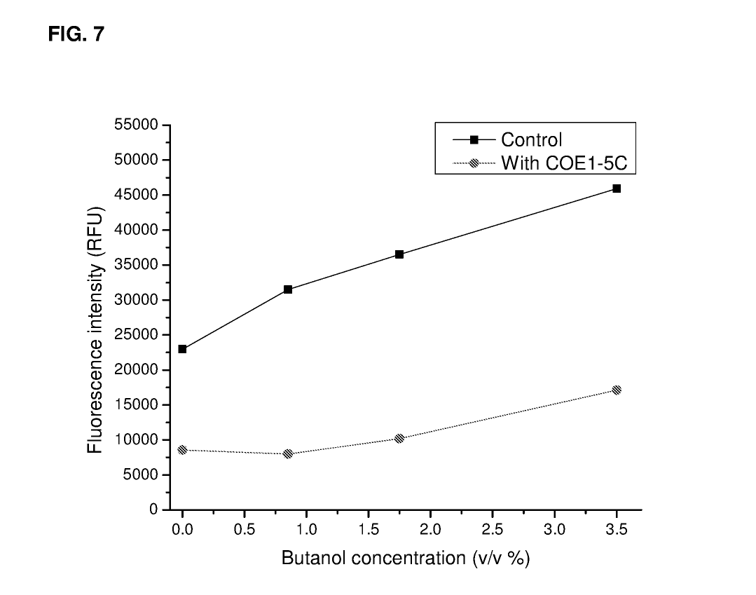

Membrane depolarization is an established means to measure membrane integrity, and has been successfully demonstrated in response to a perturbation by MIMs. Membrane depolarization of untreated E. coli K12 cells occurred in response to the butanol challenge, as indicated by an increase in fluorescence intensity (FIG. 7). However, treatment by COE1-5C mitigated the depolarization efficiency of butanol, providing experimental evidence that butanol tolerance conferred by COE1-5C is a membrane based phenomenon. COE1-5C thus modifies solvent interactions with the membrane such that trans-membrane potential is not disrupted by COE1-5C, consistent with both the findings of the single molecule tracking (FIG. 2) and the simulations (FIG. 6).

Furthermore, untreated E. coli K12 cells showed a greater tendency to partition into butanol rather than aqueous phase in a modified microbial adhesion to hydrocarbon (MATH) test. More than double the amount of E. coli K12 cells partitioned into butanol in the absence of COE1-5C relative to when COE1-5C was present, with modified MATH values of 0.35 (±0.073) and 0.17 (±0.027), respectively. This suggests a reduced affinity between the cell surface and butanol following COE1-5C treatment, consistent with the prediction from the molecular dynamics simulations that the COE1-5C retains the phospholipids within the bilayer (FIG. 6). The ability of COE1-5C to reduce the extent of lipid dissolution into the butanol phase can thus be explained in terms of increased hydrophobic interactions between the MIM and the phospholipid acyl chain which prevent them interacting with the butanol, an event that precedes the stripping out of phospholipids from the bilayer as depicted in FIG. 3.

Implications

Various natural adaptation mechanisms to arrest bacterial membrane fluidization by solvents have been described, including active transport systems that exclude solvent from cells, accelerated phospholipid biosynthesis, cell envelope fortifications and biofilm formation, amongst others.

However, it is likely that membrane modifications predominate in butanol tolerant organisms because efflux pumps have been shown to be of little importance in engineering solvent tolerance to butanol. Improving the tolerance of cell membranes to solvents could enhance multiple bioprocesses, including the production of biofuels like butanol, but also any process where either substrate, product or intermediate is toxic to membranes, e.g., the biological production of fine chemicals such as benzaldehyde.

Common interventions to improve solvent tolerance invoke genetic approaches, by assimilating molecular mechanisms for enhanced tolerance into genetically tractable host organisms like E. coli. However, genetic pathways are often complex, multiple, complementary and interdependent and solvent tolerance in E. coli has been shown to involve the differential expression of up to 270 genes. Genetic approaches, therefore, often involve more than isolating a single genetic trait and expressing it in a host organism. Because wild type E. coli produces few biofuels and fine chemicals, a double-pronged genetic approach that includes both production of and tolerance to the end product would be required. It is clear, therefore, that there is a good reason for developing alternative strategies to genetic approaches aimed at bioprocess strain improvement.

Improved butanol tolerance was demonstrated here for E. coli rather than microorganisms that naturally produce butanol—a distinction that is becoming less important with modern biotechnological advances. Using E. coli enabled a series of complementary assays to be applied focusing on a mechanistic understanding of membrane disruption by butanol and how this might be mitigated through specific structural features of the conjugated oligo-electrolyte. E. coli membranes are well-characterised and lend themselves to such a methodology. Parameterized datasets exist to model the butanol effect on the E. coli membrane and extracts of E. coli lipids are commercially available. Additionally, improvements to MIM design achieved using this framework, targeting even greater butanol tolerance in E. coli, is in itself valuable industrially due to the benefits of engineered strains of E. coli over natural butanol producers, including stability, fast growth rates and genetic tractability.

In conclusion, the examples illustrate that butanol increases phospholipid disorder in microbial membranes resulting in the dissolution of lipids into the butanol phase, causing water channels to form when lipids are displaced, culminating in the destruction of membranes at high butanol concentrations. COE1-5C mitigates the toxic effect of butanol but other MIMs such as DSSN⁺ do not. Single molecule tracking in a supported membrane bilayer suggests reduced lipid bilayer fluidity as a potential explanation for enhanced membrane stability in the presence of butanol. Furthermore, MD simulations present a mechanism by which the COE1-5C may achieve this by providing a hydrophobic support within the bilayer that fixes the acyl chains preventing the dissolution of the phospholipids into the butanol phase. The same mechanism could not be described for DSSN⁺, consistent with experimental observations with E. coli subjected to butanol challenge. The chemical modification approach described herein offers great scope for future development and exemplifies an alternative to genetic and molecular approaches to strain improvement.

By “comprising” it is meant including, but not limited to, whatever follows the word “comprising”. Thus, use of the term “comprising” indicates that the listed elements are required or mandatory, but that other elements are optional and may or may not be present.

By “consisting of” is meant including, and limited to, whatever follows the phrase “consisting of”. Thus, the phrase “consisting of” indicates that the listed elements are required or mandatory, and that no other elements may be present.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

By “about” in relation to a given numerical value, such as for temperature and period of time, it is meant to include numerical values within 10% of the specified value.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

1. A method of improving solvent tolerance in microbes, comprising:
modifying a cellular membrane of the microbes, wherein said modifying comprises contacting the microbes with a membrane insertion molecule, wherein the membrane insertion molecule comprises a conjugated oligo-electrolyte.

2. The method of claim 1, wherein the conjugated oligo-electrolyte comprises an oligo-polyphenylene vinylene conjugated oligo-electrolyte.

3. The method of claim 2, wherein the oligo-polyphenylene vinylene conjugated oligo-electrolyte comprises formula (I): 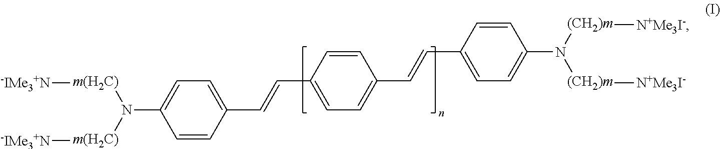
wherein m is an integer from 1 to 10;
Me is a methyl group; and
n is an integer from 1 to 5.

4. The method of claim 3, wherein in formula (I), m is an integer from 1 to 6 and n is 1, 2, or 3.

5. The method of claim 4, wherein in formula (I), m is 6 and n is 3.

6. A method of improving bio-butanol titre in a fermentation process, comprising:
modifying a cellular membrane of the microbes used in the fermentation process, wherein said modifying comprises contacting the microbes with a membrane insertion molecule, wherein the membrane insertion molecule comprises a conjugated oligo-electrolyte; and
allowing the microbes to grow in an environment comprising butanol.

7. The method of claim 6, wherein the conjugated oligo-electrolyte comprises an oligo-polyphenylene vinylene conjugated oligo-electrolyte.

8. The method of claim 7, wherein the oligo-polyphenylene vinylene conjugated oligo-electrolyte comprises formula (I): 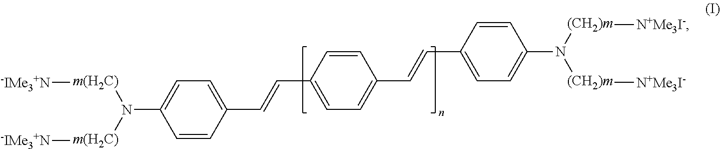
wherein m is an integer from 1 to 10;
Me is a methyl group; and
n is an integer from 1 to 5.

9. The method of claim 8, wherein in formula (I), m is an integer from 1 to 6 and n is 1, 2, or 3.

10. The method of claim 9, wherein in formula (I), m is 6 and n is 3.

11. The method of claim 6, wherein said allowing comprises allowing the microbes to grow in an environment comprising 0.85 to 8% v/v butanol.

12. The method of claim 11, wherein said allowing comprises allowing the microbes to grow in an environment comprising 3.5% v/v butanol.

13. The method of claim 11 wherein said allowing comprises allowing the microbes to grow in an environment comprising 2 to 3.5% v/v butanol.
